(19) United States
(12) Patent Application Publication
(10) Pub. No.: US 2021/0228327 A1
(43) Pub. Date: Jul. 29, 2021

(54) THREE-DIMENSIONAL PRINTED SCAFFOLD FOR CAPTURING TOXINS AND RELEASING AGENTS

(71) Applicants: THE REGENTS OF THE UNIVERSITY OF CALIFORNIA, OAKLAND, CA (US); CARBON, INC., REDWOOD CITY, CA (US)

(72) Inventors: NITASH P. BASARA, EL CERRITO, CA (US); HEE JUENG OH, Berkeley, CA (US); STEVEN W. HETTS, Hillsborough, CA (US); MARIAM S. ABOIAN, SAN FRANCISCO, CA (US); JOSEPH M. DESIMONE, REDWOOD CITY, CA (US); GREGORY R. ROBBINS, REDWOOD CITY, CA (US)

(21) Appl. No.: 17/051,686

(22) PCT Filed: Apr. 30, 2019
(86) PCT No.: PCT/US19/29979

§ 371 Date: Oct. 29, 2020

Related U.S. Application Data
(60) Provisional application No. 62/664,508, filed on Apr. 30, 2018.

(51) Int. Cl. A61F 2/01; B01J 20/26; B01J 20/28; B01J 20/30; B01J 20/32; B33Y 40/20; B33Y 70/00; B33Y 80/00

(52) U.S. Cl. CPC A61F 2/0103; A61F 2/011; B01J 20/262; B01J 20/28042; B01J 20/3007; B33Y 10/00; B01J 20/3289; B33Y 40/20; B33Y 70/00; B33Y 80/00; A61F 2230/0067; B01J 20/3285

(57) ABSTRACT

A chemical absorber to absorb and release compounds includes a porous scaffold of lattices, modified surfaces of the scaffold, wherein the modification is selected based upon an ability to bond with or release a particular compound, and a center hole in the scaffold to accommodate a guide wire.

### RELATED APPLICATIONS

This application claims priority to U.S. Provisional Patent Application 62/664,508, filed Apr. 30, 2018, which is incorporated by reference here in its entirety.

### GOVERNMENT SUPPORT

This invention was made with government support under Contract/Grant Numbers CA194533 and EB012331 awarded by the National Institutes of Health and Contact/Grant Numbers DE-ACO2-05CH11231 and DE-ACO2-05CH11232 awarded by the Department of Energy. The government has certain rights in the invention.

### BACKGROUND

Medications for treatment of cancer, infection, thrombosis, and other diseases, are commonly very toxic and are therefore effective at the location of the tumor or infection, but toxic to normal tissues, resulting in significant side effects. Despite efforts to develop increasingly targeted and personalized therapeutics, system toxic side effects limit the use of specific medications and dosing of many medications for treatment of cancer, infection, and thrombolysis. One of the methods to limit toxicity common in medical practice for specific types of cancer is direct infusion of chemotherapy into the feeding artery supplying the tumor. This limits the systemic exposure of the toxic chemical and directs the chemotherapy to the region where treatment is needed.

Unfortunately, more than 50-80% of the injected drug is not trapped in the target organ and bypasses the tumor to general circulation. These toxic chemicals then enter the body and can cause side effects, which may include irreversible cardiac failure, fatigue, hair loss, bruising and bleeding, infection, anemia, nausea and vomiting, appetite changes, digestive disruptions, weight changes, cognitive abilities effect, mood changes, etc.

### BRIEF DESCRIPTION OF THE DRAWINGS

FIGS. 1A-1C show diagrams of an embodiment of the absorber, the chemical structure of a chemotherapy drug, and a schematic of an endovascular treatment of live cancer.

FIGS. 2A-2C show an embodiment of a three-dimensional printed porous cylinder, optical micrographs of a three-dimensional printed porous cylinder, and chemical reactions used in a three-dimensional printer.

FIG. 3 show a chemical structure of a block copolymer usable with the embodiments

FIG. 4 shows a schematic of in vivo experiments.

FIGS. 5A-5B show fluoroscopy images of absorbers taken during in vivo experiments.

FIGS. 6A-6D show schematics of the location for placement of absorbers, concentrations of the chemotherapy drug in different sampling locations for each absorber, and photographs of plasma from both control absorbers and coated absorbers.

FIGS. 7A-7B show photographs of mixtures after addition of crushed absorbers used during in vivo experiments.

### DETAILED DESCRIPTION OF THE EMBODIMENTS

Industries routinely use absorption columns to remove pollutants from chemical streams. Embodiments here include porous absorbers for capturing excess chemotherapy drugs that do not become absorbed by the target tumor. The embodiments here introduce an absorber into the draining vein and then the absorber removes a significant fraction of the injected chemotherapy drugs. The absorber can also remove significant amounts of different types of drugs, such as anti-microbials, thrombolytic agents, toxins from bacterial infections, environmental toxins, or cells, may be captured, or bound, using specific chemical, physical biological, and any combinations thereof using different features of the three-dimensional, printed absorbers. In addition, the absorbers can capture/remove unwanted molecules/materials in the body which the natural systems in the body do not properly function to remove them, and result in chronic health issues. For example, lactic acid could be removed from the blood of patients with acute lactic acidosis.

The embodiments here also include porous materials for releasing drugs at the target location at a constant rate required for the condition of patients. The embodiments here also include a system to release the drug at the target upstream location and capture the drug at the downstream location. One should note that while the apparatus is referred to as an absorber, it also may act as a delivery system to release drugs, such as nano-particles or micro-particles of therapeutic compounds and other agents for use in health care. The term absorber encompasses both the release and absorption aspects of the embodiments.

While the discussion below focuses on chemotherapy drugs, the application of the absorbers to remove other harmful materials from the body should not be limited to this. The term ‘toxin’ as used here means any material that is toxic to the human body, even if it is used to treat tumors or other illnesses. The toxin is harmful to other parts of the body. The examples of toxins include candidate chemotherapeutics.

Candidate chemotherapeutics include, but are not limited to alkylating agents: bifunctional alkylators (Cyclophosphamide, Mechlorethamine, Chlorambucil, Melphalan), and monofunctional alkylators (Dacarbazine, Nitrosoureas, Temozolomide), (using DNA strands); anthracyclines: doxorubicin, daunorubicin, dipirubicin, iadrubicin, mitoxantrone, and valrubicin (using ion-exchange functional groups); cytoskeletal disruptors: pacilaxel, docetaxel, abraxane, and taxotere (using protein (whole protein or subunit of protein) based capture; Epothilones; Histone deacetylase inhibitors (HDAC): vorinostat, and romidepsin (using protein and/or DNA based capture); Inhibitors of topoisomerase I: irinotecan, and topotecan (using protein and/or DNA based capture); Inhibitors of topoisomerase II: etoposide, teniposide, and tafluposide (using protein and/or DNA based capture); Nucleotide analogs: hydroxyurea, anacitidine, capecitabine, cytarabind, and doxifluridine, etc. (using protein and/or DNA based capture); Peptide antibiotics: bleomycin, and actinomycin; Platinum-based agents: carboplatin, cisplatin, and oxaliplatin (using DNA strands); Retinoids; Vinca alkaloids and derivatives: vinblastine, vincristine, vindesine, and vinorelbine (using protein and/or DNA based capture).

The term ‘vessel’ as used here means any vessel into which the absorber is placed. Typically, this will be the vein that drains the body structure, such as an organ, but could be placed into other vessels as well. In addition, the absorbers can be located in organ, such as in fatty layers of organs, or can be designed to be deployed in any location of the body if necessary.

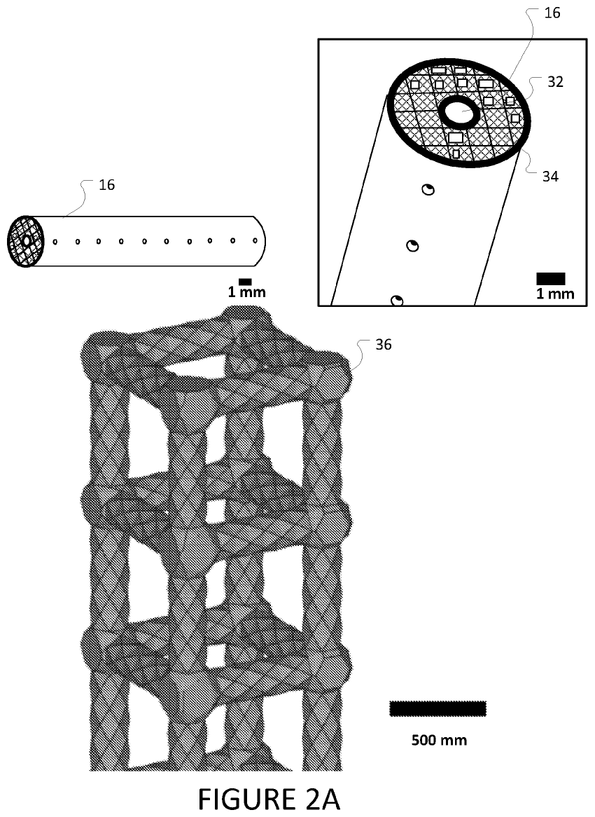

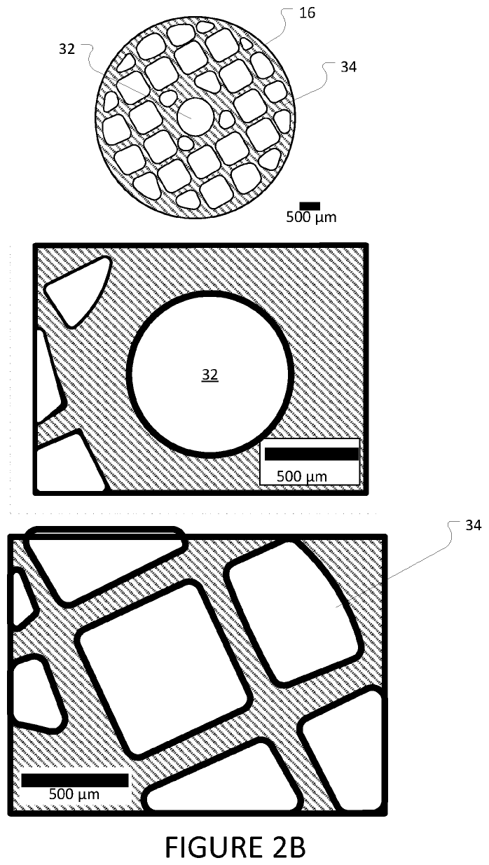

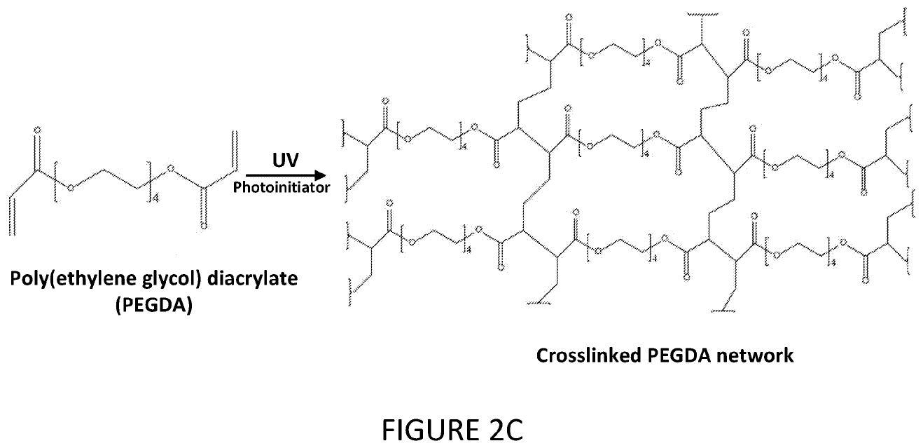

As used here, the term “lattice” is a structure that forms a unit cell of a repeating structure. In FIG. 2A, the bottom drawing shows a columnar formation 36 of cubical structures. In this embodiment, one of those cubical structures is a lattice. The columnar structure, which may have multiple columns, is referred to here as a scaffold. A scaffold consists of a structure that supports another functional material(s) on the surface to have chemical and/or physical interaction(s) with target molecules such as a drug, DNA, protein, etc. In the embodiments here, the scaffold receives a coating. Also a scaffold can be made of functional material(s) or additional surface modification can be made on the surface layer. The term “cylinder” as used here is the shape of the scaffold that was 3D printed. The scaffold consists of a network of lattices 34.

Generally, the absorber consists of a cylinder having within it a scaffold or lattice of material. The shape of the absorber/releaser is dictated by the shape of the blood vessel at the target location of the patient. This can be determined for each individual patient by methods such as MRI and printed in accordance to the specifics of the patient. The scaffold or lattice has a coating or other surface modification that absorbs the toxin. Because it can be printed, the cylinder could have any circumference or length, customizable to the individual user's vessels.

The scaffold or lattice surfaces can be modified using different chemical reactions, such as polymerization, catalytic reactions, surface coating, etching, surface modification such as a dopamine coating, cross-linkage, etc., to introduce functional groups that can capture/bind to target toxins. Functional groups can be strong or weak cation exchange groups, strong or weak anion exchange groups, DNA strands, biological ligands, proteins, antibodies, enzymes, nano-particles, micro-particles, magnetic particles, etc. depending upon the target molecules. Magnetic particles are used for imaging with NMR and/or MRI. The removal of such magnetic particles after operation is important for safety.

In addition, the absorbing or releasing coating could be formed by many different kinds of polymers. For example, one possibility could consist of block copolymers wherein one of the blocks contains the active functional groups above, while the other causes the polymer to adhere to the scaffold. Other possibilities include random copolymers comprising functional and adhering monomers, and graft copolymers comprising functional and adhering monomers.

The surface of the absorbers can be designed to capture multiple drugs to be used with drug cocktails that are commonly used in cancer chemotherapy. The absorber surface can be printed or modified to have multiple layers of different materials to facilitate the capacity and rate of drug capture, and the capture the mixtures of drugs and target molecules. Absorbers can be prepared using elastomeric materials with controlled mechanical properties. The surface modification is selected based upon its ability to bond with the particular target molecules, either for release or capture. The target molecules, whether for release or capture, whether drugs or nano-particles or micro-particles, will be referred to here as “compounds.”

Absorbers with different designs and coatings can be deployed at the same time or in rapid succession to affect multiple toxins, drugs, or processes.

The system may incorporate the absorber and other related parts. The absorbers can incorporate guide wires, an introducing sheath, and/or other related devices, such as balloons for easier operation with less blood loss. The absorbers can be compressible and stretchable to fill the vessels of interest and for easier operation. The absorbers of different chemical formula and/or mechanical properties can be assembled in desired locations to optimize their binding/releasing abilities to target molecules, not interfere with the blood flow, and/or manipulate the blood flow to a desirable rate. Device design features including device shape and balloons can also be used to alter blood flow adjacent to the device to improve drug binding or release.

The dimensions, shape, and mechanical properties of the absorber can be carefully modified to manipulate the blood flow to a desirable rate, in terms of volumetric flow rate, blood flow residence time contacting the absorbers, circulating the blood flow in the absorbers, etc. Such dimensions, shape and mechanical properties of the absorbers can be determined by the size of vessels, location, and the blood flow rate in the desired location. The absorbers can be used as balloons or stents to construct the vessel or organs in which a structures is destroyed or collapsed, such as in an aneurism, and/or slow down the blood flow.

Regarding the incorporation of sheath and guide wires with the absorbers by using 3D printing, the sheath, guidewire, and/or other necessary parts for operation can be prepared by special chemical formula to make these parts function as the absorbers well. The inside and outside of sheath, guidewire, and/or other parts can be modified, such as by surface modification, to have special functional groups to bind/release target molecules for drug capture/drug release purposes. The whole system consisting of sheath, guidewire, and other parts, which may include catheters, connecting parts, etc., may be referred to here as “the absorber/releasing system.” Certain designs of porous scaffolds may be manufactured by more conventional polymer processing methods such as injection molding.

The following discussion focuses on chemotherapy with the understanding that this is merely for discussion purposes only. Cancer is becoming the leading cause of death in most westernized nations. Although there have been enormous efforts to develop more targeted and personalized cancer therapeutics, dosing of drugs in cancer chemotherapy is often limited by systemic toxic side effects. During intra-arterial chemotherapy infusion to a target organ, excess drug not trapped in the target organ passes through to the veins draining the organ, and then circulates to the rest of the body, causing toxicities in distant locations. Typically, more than 50-80% of the injected drug is not trapped in the target organ and bypasses the tumor to general circulation.

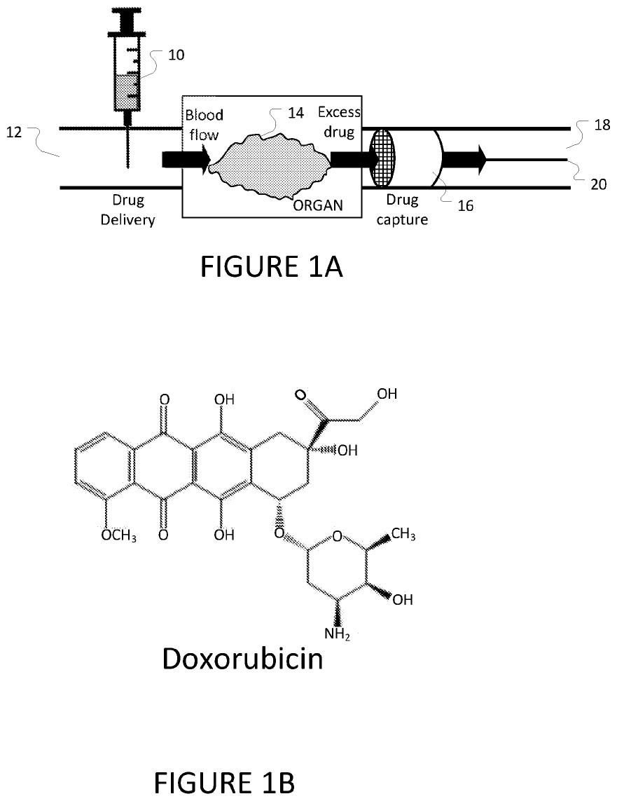

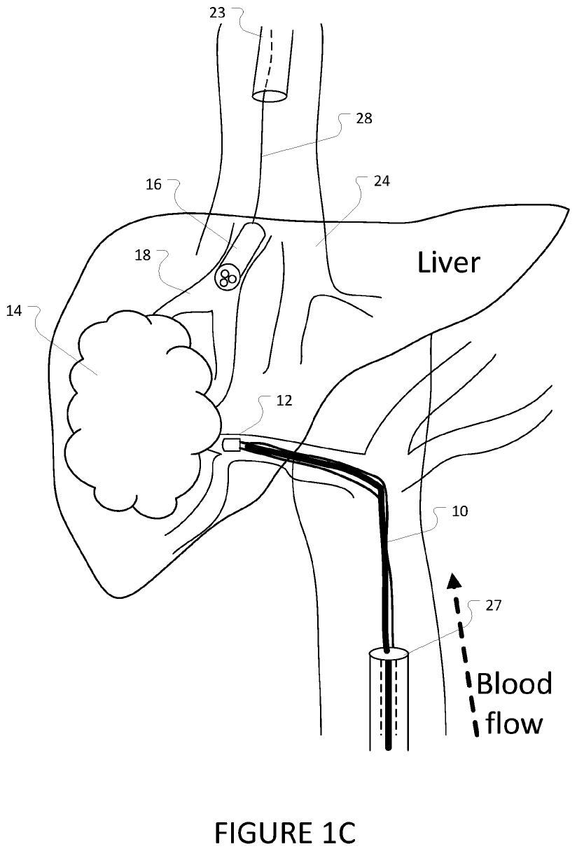

In the context of reducing the toxicity of chemotherapy, the embodiments present the development of a new biomedical device: an absorber that captures excess chemotherapeutic drug before it is released in the body. This absorber is temporarily deployed in the vein draining the organ undergoing intra-arterial chemotherapy infusion, and is removed after the infusion is completed. FIG. 1A depicts this schematically, showing the treatment of a tumor within the liver. The drug 10 is injected in the artery 12, in this case the hepatic artery, as is the case in conventional intra-arterial chemotherapy infusion. The blood exiting the organ, in this example the liver, through the draining vein 18, in this case the hepatic vein, passes through the absorber 16 that captures the excess drug, resulting in drug-free blood 20. The particular drug used in this study is doxorubicin.

FIG. 1B shows the chemical structure of doxorubicin. The proposed approach for doxorubicin capture is shown in FIG. 1C. Minimally invasive image-guided endovascular surgical procedures are used to deliver the drug 10 to the tumor 14 using the hepatic artery 12, and to place the absorber 16 in the hepatic vein 14, hepatic vein confluence, or suprahepatic inferior vena cava 24. The standard introducer sheaths, such as 23 and 27, and guide wires such as 28 used to accomplish this task are shown in FIG. 1C. The approach described in FIGS. 1A-1C can be used to minimize toxicity effects of chemotherapy used at different locations in the body. The toxicity of drugs used to treat other diseases besides cancer may also be modulated by the proposed approach. Similarly, toxins from bacterial infections, environmental toxins, or cells themselves could be captured using specific chemical, physical, or biological features.

Doxorubicin is a low-cost, highly effective agent frequently used in chemotherapy for several decades. Based on a linear dose response model, increasing the dose of doxorubicin linearly increases tumor cell death. This provides motivation for higher-dose doxorubicin therapy, but the side effects of high dose doxorubicin therapy include irreversible cardiac failure, which limits implementation of the high dose regimen. An established and highly effective agent like doxorubicin is a compelling first candidate for demonstrating the proposed drug capture approach.

For the absorber to work efficiently in the embodiments using doxorubicin in liver infusion chemotherapy, it must selectively bind the target drug within an hour or less. The structure of the absorber must be carefully designed and fabricated so as not to severely impair blood flow or cause thrombosis, although patients are usually anticoagulated during interventional radiology procedures limiting thrombosis. Custom-made absorbers must be used as individual patients have veins of different dimensions. The inventors have used 3D printing to fabricate the absorbers used in this study. Successful design, fabrication and deployment of the absorber has the potential to open a new route to help patients fight cancer.

Porous cylinders, shown in FIGS. 2A-C, were printed. The absorbers were 5 mm in diameter and 30 mm in length. The targeted internal structure of the cylindrical absorber 16 is shown in FIGS. 2A-2B. A central hole 32 that runs through the cylinder enables attachment of a device to a guide wire needed for minimally invasive surgery. In one embodiment, the central hole has a diameter of 0.89 mm, and the dimensions of the central hole can be changed if necessary. This is surrounded by a square lattice structure 34 with a characteristic dimension of 800 μm, with the cylinder acting as a scaffold for the lattice. This dimension was chosen to prevent hemolysis of blood cells; white blood cells, with diameters about 9-20 μm, are the largest component of blood. The porous cylinders were printed by photo-induced crosslinking of poly(ethylene glycol) diacrylate (PEGDA), shown in FIG. 2C.

Poly(ethylene glycol)-based polymers are widely used in biomedical engineering because of their biocompatibility and fouling resistance. Moreover, other relevant properties such as mechanical strength and water swelling of the PEG based polymers can be readily tuned by controlling the polymerization conditions. Optical micrographs of the 3D printed porous cylinders are shown in FIG. 2B. It is clear that the printing process faithfully reproduces the targeted internal structures shown in FIG. 2A. The porous cylinder serves as the scaffold of the absorber 16.

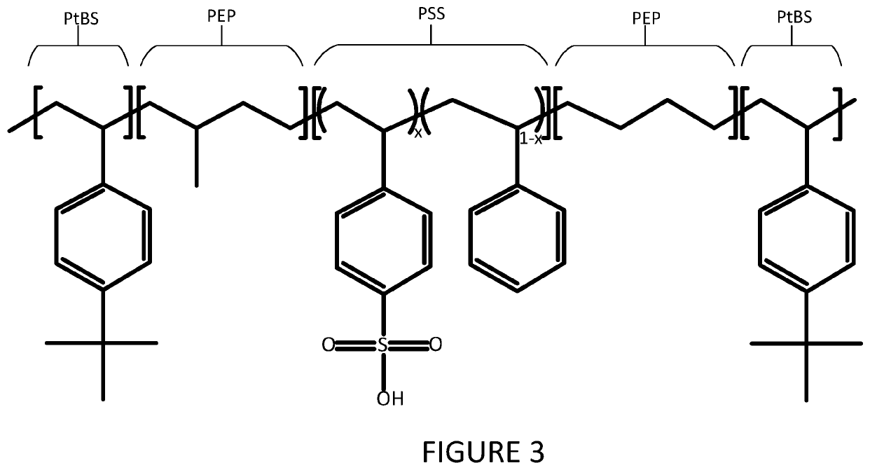

The surface of the porous cylinders was coated with a poly(tert-butylstyrene)-b-poly(ethylene-co-propylene)-b-poly(styrene-co-styrene sulfonate)-b-poly(ethylene-co-propylene)-b-poly(tert-butylstyrene) (PtBS-PEP-PSS-PEP-PtBS) block polymer provided by Kraton Performance Polymers, Inc. (Houston, Tex.). The chemical structure of block copolymer is shown in FIG. 3. The block copolymer was provided in the form of 10 wt % solution of the polymer dissolved in a mixture of heptane and cyclohexane (72:28 by mass). The 3D printed cylinders were fit into silicone tubing and the polymer solution was pumped through the cylinders for 10 min. The cylinders were then dried first in air at 50° C. for 1 hour and 30 minutes, followed by drying under vacuum at room temperature for 24 hrs. This resulted in a coating of the copolymer on the printed cylinders. To visualize this coating, the surface-modified cylinders were imaged using X-ray microtomography. The coating thickness is more-or-less uniform, ranging from 30 to 50 μm.

In one embodiment, the choice for the polymer coating was informed by previous studies where it was shown that polystyrenesulfonate chains demonstrated high capacity for binding with doxorubicin. It is likely that the PtBS and PEP blocks in the block copolymer are responsible for adhesion between the coating and 3D printed scaffold. The approach for coating the cylinders described here was arrived at after considerable trial and error. Small changes in the composition of either the block copolymer or the solvent result in unstable coating on the scaffolds.

In vivo experiments were performed with the coated 3D printed absorbers described above in three animal models (swine). The diameter of the absorbers (5 mm) was determined by the size of the introducer sheath, in one embodiment the sheath consisted of an 18 French or 6 mm diameter sheath that could be accommodated in the common femoral and common iliac veins of the swine. These are similar in diameter to the hepatic veins in an adult human.

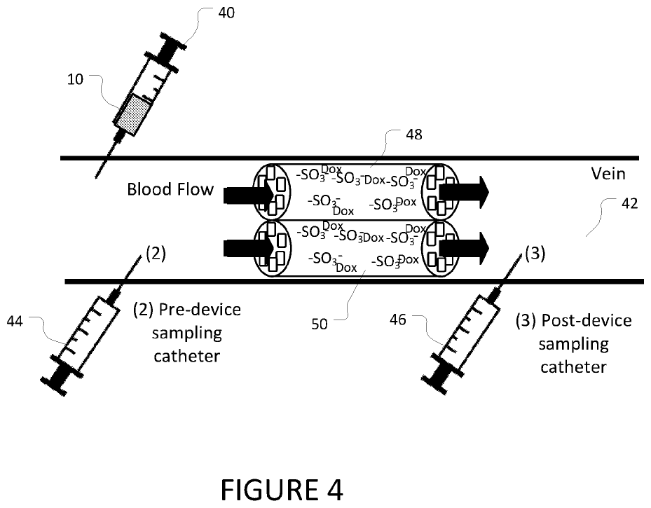

The diameter of the introducer sheath is minimized to minimize blood loss during the operation. The length of the absorbers (30 mm) was chosen to match the length of the common iliac vein. The common iliac vein 42 was chosen to facilitate interpretation of experimental data and demonstrate proof-of-concept. Also, the diameter of the common iliac vein is approximately 10 mm, like the diameter of human hepatic veins near their confluence with the inferior vena cava where the absorbers will be placed for capturing excess drug draining the liver during hepatic intra-arterial chemotherapy infusion as shown in see FIG. 1C. To minimize the blood flow around the absorber, two cylinders 48 and 50 were brought to the desired location using the introducer sheath, one after the other, and arranged in parallel as shown in FIG. 4.

The absorbers were tested in the swine models undergoing chemo-infusion in the common iliac vein 42 of 50 mg of Doxorubicin over 10 min, corresponding to a typical dose used clinically in chemotherapy for intra-arterial treatment of hepatocellular carcinoma. Doxorubicin concentrations were monitored as a function of time using blood-sampling catheters at three different locations. Two locations, the pre-device 44 and post-device 46 sampling catheters, are depicted schematically in FIG. 4. The pre-device catheter is located between the injection catheter 40 and the absorber. The post-device catheter is located just after the absorber. The third catheter, not shown here, was located at the internal jugular vein, well-removed from the common iliac vein such that any blood sample taken from this location will reflect the systemic drug concentration, as doxorubicin would have had to pass through the inferior vena cava, heart, pulmonary vasculature, systemic arteries, capillaries, and systemic veins to reach that sampling point. The discussion here refers this as the peripheral location.

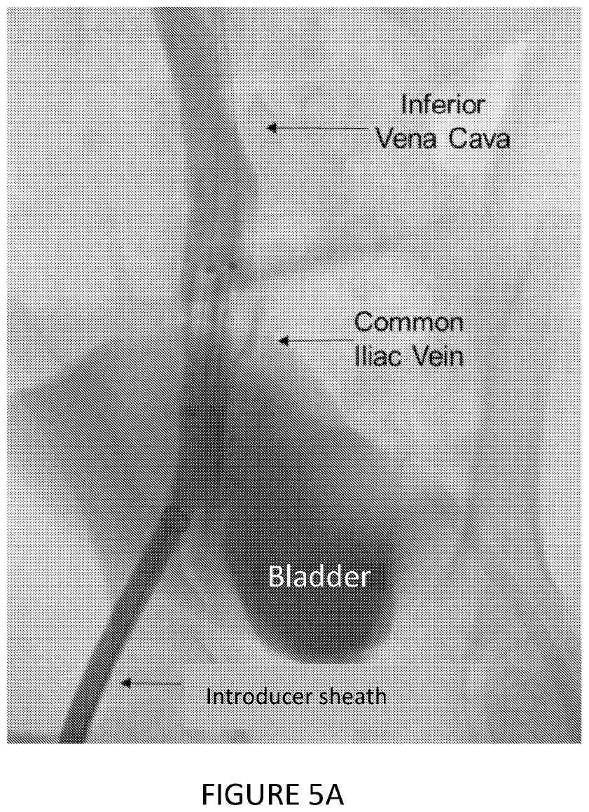

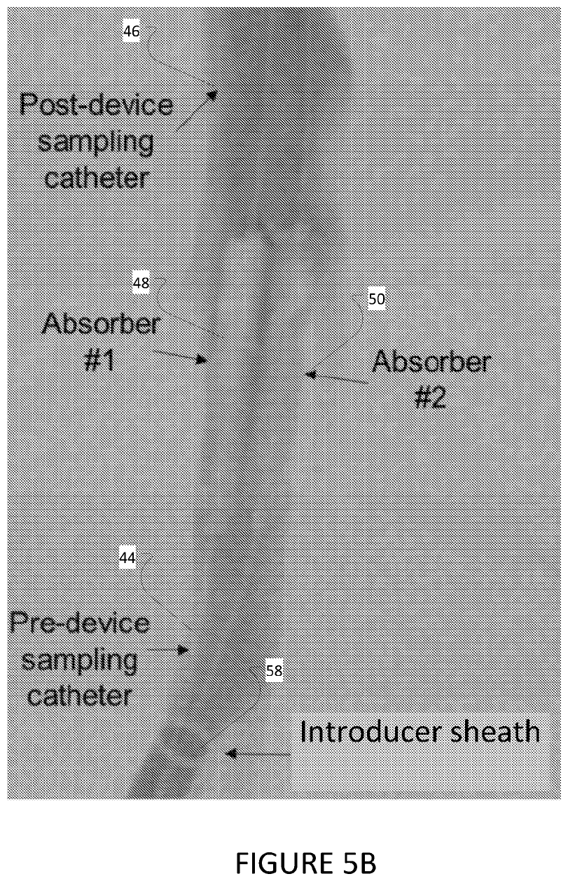

X-ray fluoroscopy images of the absorbers in the common iliac vein obtained during one of the in vivo experiments are shown in FIGS. 5A-5B. The introduction sheath and guide wires used to deliver the absorbers are clearly seen in FIG. 5A. The sheath was introduced via a common femoral vein. The absorbers are located between metallic fasteners that are also visible in FIG. 5A. The higher magnification image of FIG. 5B shows the two absorbers 48, and 50, arranged in parallel.

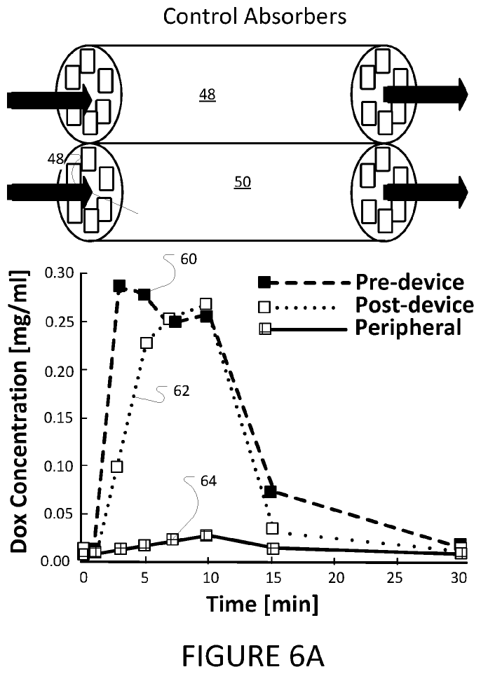

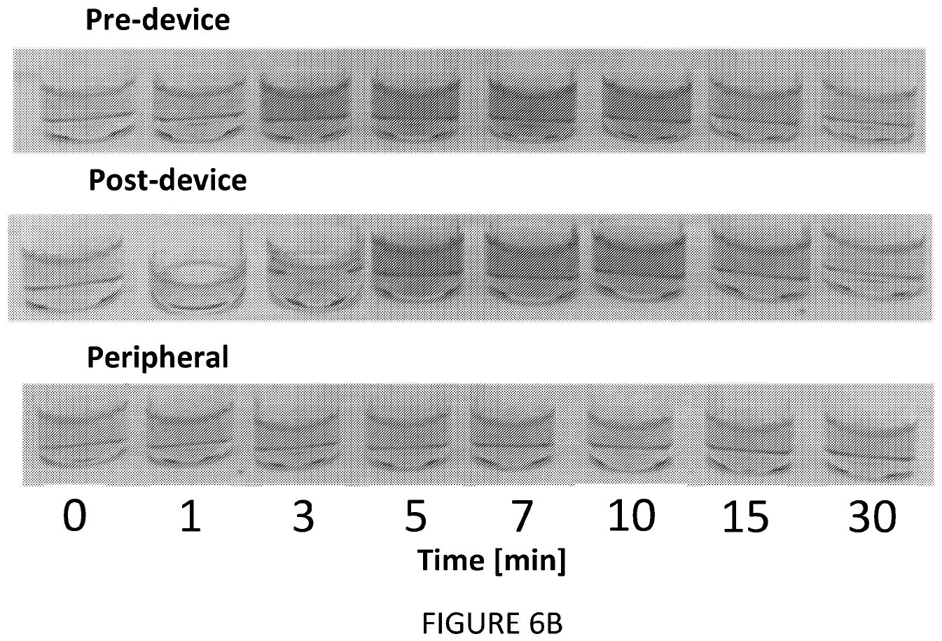

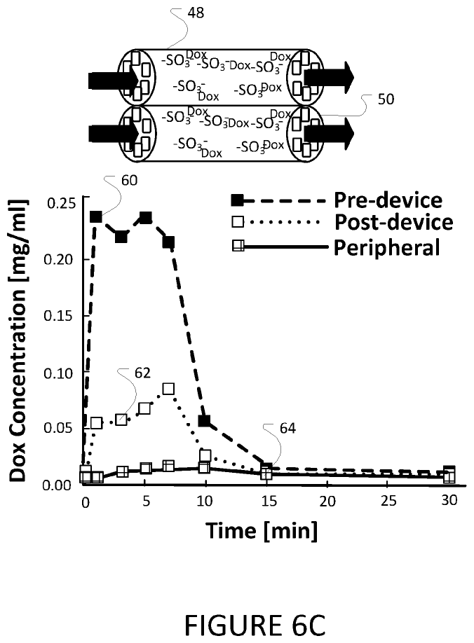

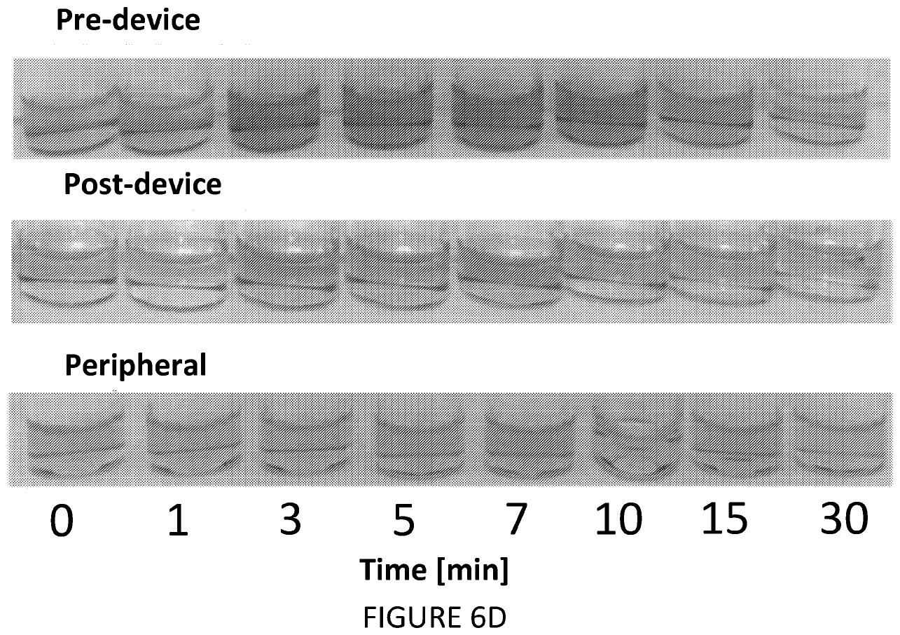

Results of two separate in vivo experiments are shown in FIGS. 6A-D. FIG. 6A shows the measured doxorubicin concentration as a function of time at the three locations described above during a control experiment, wherein uncoated absorbers were placed in common iliac vein. The doxorubicin concentration measured at the pre-device, show at line 60, and the post-device, shown at line 62, locations are qualitatively similar, indicating that most of the doxorubicin injected passes through the absorbers. In both cases, the doxorubicin concentration increases rapidly during the first 3 min, stays constant about 5 min, and then decreases to zero in about 30 min. The doxorubicin concentration measured at the peripheral location shown at line 64 increases only slightly when doxorubicin is injected into the animal model. FIG. 6B shows the images of the plasma from the centrifuged samples obtained from three sampling catheters during the control experiments. Since doxorubicin has a characteristic orange color, the higher the doxorubicin concentration is, the darker the orange color is in the samples, which translates to darker shades of gray in the depiction. The color darkness in the samples is qualitatively consistent with the doxorubicin concentration profiles shown in FIG. 6A. There is little qualitative difference between the images obtained from the pre-device and the post-device catheters in the control experiment.

FIG. 6C shows the measured doxorubicin concentration as a function of time when coated absorbers were deployed. These results differ significantly from those in FIG. 6A. FIG. 6C shows the post-device doxorubicin concentration at line 62, and the peripheral location at line 64, is significantly lower than that measured at the pre-device location at line 60. The integrated areas under the two data sets enable quantification of the drug capture efficacy.

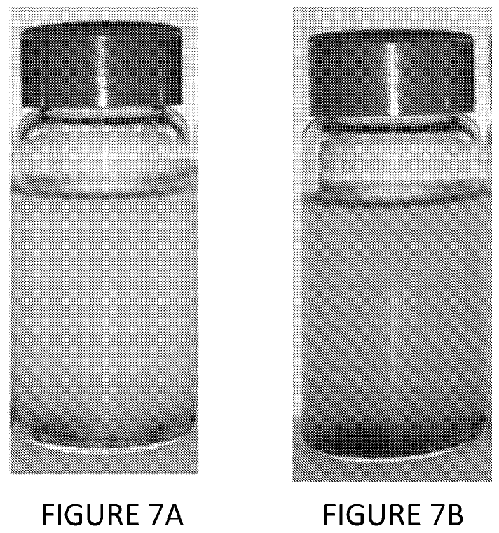

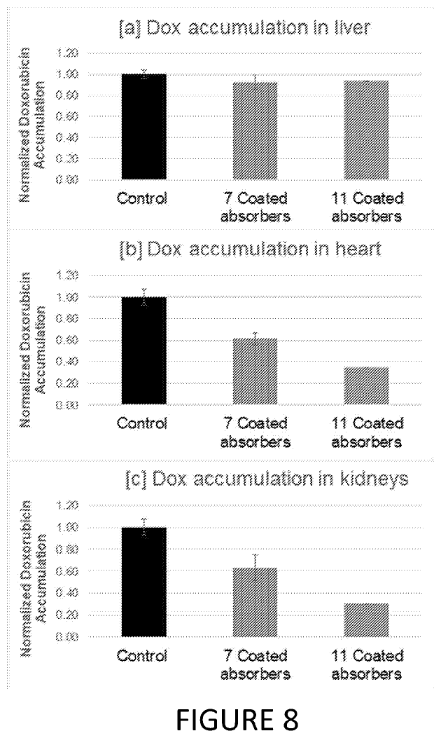

In this experiment, 69% of the doxorubicin is captured by the coated 3D printed absorbers. The images of the plasma from the centrifuged samples obtained from three sampling catheters during this experiment, shown in FIG. 6D, confirm the removal of doxorubicin. After the in vivo experiments, the absorbers were crushed and immersed in an aqueous mixture of potassium chloride and ethanol, in one embodiment 20% w/v to diffuse out doxorubicin from the absorbers. A colorless solution was obtained when the uncoated absorbers, used in in vivo experiments, were studied, as shown in FIG. 7A. In contrast, an orange colored solution, translated into shade of gray here, was obtained when the coated absorbers, used in in vivo experiments, were studied, as shown in FIG. 7B.

The experiments suggest doxorubicin binds to the absorbers irreversibly; the inventors tried to release the doxorubicin by pumping the aqueous potassium chloride and ethanol mixture described above past the filters for one month. Analysis of the mixture showed negligible doxorubicin concentrations (less than 0.001 mg/ml). The images shown in FIG. 7B, obtained after this procedure, suggests that the absorbed doxorubicin binds strongly to the scaffolds after it diffuses through the coating layer. After completing the in vivo experiments, the absorbers were reanalyzed using X-ray microtomography. The microtomography images obtained were like those shown in FIG. 3B, indicating that the coating layer was stable during the drug capture process. In addition, problems related to blood clots and other biocompatibility issues were not observed during the operation.

The in vivo drug capture experiments were repeated four times. The results of the other three experiments were like those reported in FIGS. 6A-D. The doxorubicin capture efficacy ranging from 57 to 69%. The results are shown in supplementary materials.

The embodiments here include designed, built, and deployed porous absorbers for capturing chemotherapy drugs in vivo before they are released in the body to reduce systemic toxic side effects. The porosity was obtained by 3D printing of the lattice structure within the cylinders. The application of a polystyrenesulfonate coating on the absorber was essential for drug capture. The initial design enables the capture of 69% of the administered drug without noticeable adverse side effects.

Numerous approaches for using the platform have been developed to improve the efficacy of drug capture. Most simply, the number of absorber devices could be increased, increasing total surface area for drug biding. The lattice size could be decreased to enhance drug capture. Additional improvement in performance may be obtained by changing the chemical composition and thickness of the coating layer or by changing the lattice structure such as from cubic to hexagonal or by making a non-uniform lattice with larger pores in the front. The lattice may be of different geometries, they could be cube-like or hexagonal, of be quasi-periodic structures like a quasicrystal, or an aperiodic structure with different geometries at the front and back. The geometry of the lattice could change continuously either radially or axially with wider struts at some locations and narrower struts at other locations.

In future clinical trials one may use custom 3D printed elastomeric absorbers with patient-specific form factors that fit optimally in the vein(s) of the patient, as can be created from pre-procedure Computed Tomography (CT) or Magnetic Resonance Imaging (MRI) datasets.

### Example 1

Cylindrical porous absorbers for this study were prepared at Carbon, Inc., a 3D printing company located at Redwood City, Calif., USA. The prepolymer solution was prepared by adding 1 wt % initiators (i.e., 0.8 wt % of 2, 4, 6-Trimethylbenzoyl-diphenylphosphine oxide (TPO, Sigma Aldrich, USA) and 0.2 wt % of 2-Isopropylthioxanthone (ITX, Esstech, Inc., USA) and 0.23 wt % of carbon black pigment to poly(ethylene glycol) diacrylate (PEGDA, MW=250 g/mol, Sigma Aldrich, USA.) (see FIG. 2C). The solution was photo-polymerized by using the Continuous Liquid Interface Production (CLIP) method. The cylinders obtained by this process were washed in 2-propanol to wash away uncured resin from the polymer network. The cylinders were allowed to air dry after washing and were UV post-cured using a Dymax ECE 5000 UV cure chamber (Torrington, Conn., USA) in 30 second intervals with rotation in-between cures for a total of 2 mins. Absorbers were imaged measured using a Keyence VHX-5000 microscope (Itasca, Ill., USA).

The surface of the 3D printed porous cylinders was modified by coating a thin layer of sulfonated styrenic pentablock copolymers. The sulfonated styrenic pentablock copolymers (PtBS-PEP-PSS-PEP-PtBS) were synthesized via anionic polymerization and a subsequent post-polymerization sulfonation process, and detailed procedures have been described elsewhere. The sulfonation level (mol %) of the middle polystyrene (PS) block was controlled to a desired ion exchange capacity (IEC). In this study, the sulfonated pentablock copolymer of the IEC=2.0 meq/g (dry polymer) (sulfonation level=52 mol %) was used. The number average molecular weight of unsulfonated pentablock copolymer is approximately 78,000 g/mol (block mass fractions are PtBS:PEP:PSS=0.33:0.27:0.40), and the volume fraction of mid PSS block is 0.434.

The uncoated and coated absorbers were imaged using synchrotron hard X-ray microtomography at beamline 8.3.2. of the Advanced Light Source at Lawrence Berkeley National Laboratory. X-rays with energies ranging from 12-25 keV were generated by the synchrotron and illuminated the sample. The X-ray shadow transmitted through the sample was converted using a scintillator into visible light. This image was magnified by an optical microscope and converted into a digital image file. As the sample was rotated through 180° by a fraction of a degree, a total of 1,313˜2016 images were collected. These projection images were reconstructed using the program Xi-Cam to cross-sectional slice images, and subsequently stacked to generate 3D reconstructed images of the 397 cylinders.

3D printed absorbers were tested in vivo in three swine models (40-45 kg). The absorber was strung along a polytetrafluoroethylene (PTFE) coated nitinol guide wire (Glidewire®, Terumo Interventional Systems, Somerset, N.J., USA) for smooth and rapid movement through tortuous blood vessels; The guide wire went through the middle hole of the absorber, and two metallic fasteners on each end of the absorber were used to keep the absorber in place.

In vivo experiments were performed under compliance with the protocols of the Institutional Animal Care and Use Committee (IACUC) at the University of California, San Francisco (UCSF). Each animal was monitored with blood pressure, pulse oximetry, heart rate, and electrocardiogram while under general anesthesia with isoflurane. An 18 French (diameter=6 mm) introducer sheath placed into the common femoral vein was used to deliver the absorbers into the common iliac vein. Sampling and injection catheters were placed under fluoroscopy guidance at the spot of interest relative to the absorbers. Pre-device sampling catheter was introduced via the sheath to the left common iliac vein. Post-device sampling catheter was introduced through the internal jugular vein and was placed into the common iliac vein adjacent to the bifurcation of the vena cava. The distances between the catheters and absorbers were carefully adjusted to be consistent over a series of in vivo experiments. Prior to the start of the experiments, patency of the venous system was demonstrated using iodinated contrast injection (iohexol, Omnipaque-300, GE Healthcare, USA).

To simulate intra-arterial chemotherapy dosing, 50 mg of Doxorubicin (2 mg/ml, Doxorubicin 419 hydrochloride Injection, United States Pharmacopeia, Pfizer, New York, N.Y., USA) was injected into the common iliac vein via an infusion pump at a constant rate of 2.5 ml/min over 10 min. Blood aliquots of 2 ml at different times from the pre-device, post-device, and peripheral sampling locations were collected after 1.5 ml of blood was wasted to account for the volume within the catheter.

Doxorubicin concentrations in the blood aliquots were determined using fluorescence spectroscopy. Fluorescence measurements were made using a FlexStation 3 Multi-Mode Microplate Reader (Molecular Devices, San Jose, Calif.) at a known emission wavelength of 550 nm upon excitation with a 480 nm laser. The doxorubicin concentration was calculated from the measured fluorescence at 550 nm using the calibration curve, which correlates fluorescence emission to doxorubicin concentration.

### Example 2

The coated and uncoated absorbers of Example 1 were used in additional in vivo experiments were performed on four pigs by deploying the multiple coated absorbers in the hepatic veins and Inferior Vena Cava (IVC) of the animals as depicted in FIG. 1C. The pigs underwent 10 minute intra-arterial infusion of doxorubicin (200 mg) into the common hepatic artery to mimic clinical TACE (transarterial chemoembolization) procedures. After euthanasia, doxorubicin concentrations in organ tissues were analyzed. During the in vivo experiments, no hemodynamic, thrombotic, or immunological complications were observed.

Doxorubicin concentrations in liver, heart, and kidneys were measured to evaluate the reduction of doxorubicin accumulation in major organs. Control experiments were performed on three pigs where no absorber was deployed but the pigs underwent the same intra-arterial infusion of doxorubicin (200 mg) as described above. For easier comparison, the doxorubicin concentrations in the organs of the control experiments were used to normalize the doxorubicin concentrations in those of the in vivo experiments where the coated absorbers were deployed.

The result of organ tissue analysis is shown in FIG. 8. For the liver, the amounts of accumulated doxorubicin in the control experiments and the in vivo experiments are similar, as expected. However, significant reduction in doxorubicin accumulation in the heart (40% reduction with 7 coated absorbers, and 65% reduction with 11 coated absorbers) and in the kidneys (37% reduction with 7 coated absorbers, and 70% reduction with 11 coated absorbers) are observed by deploying the coated absorbers in the hepatic veins and/or the IVC. As the number of coated absorbers increases (i.e. the surface area of the coated absorbers increases), the doxorubicin accumulation in the heart and kidneys decreases. Other organs such as spleen and lung also show a similar trend but are not shown here for brevity.

The above discussion focused on removal of excess chemotherapy drugs in an infusion treatment. As mentioned previously, this is just one example of an application and is not intended to limit the scope of the claims. Other types of medications and substances may be removed using these absorbers. Further, the techniques and structure of the absorbers may be used to release localized medications or other therapeutic agents after insertion.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A chemical absorber to absorb and release compounds, comprising:
a porous scaffold of lattices;
a surface modification on at least one surface of the lattices, wherein the surface modification is selected based upon an ability to one of either bond with, or release, a particular compound; and
a center hole in the scaffold to accommodate a guide wire.

2. The chemical absorber as claimed in claim 1, wherein the absorber is comprised of elastomeric materials that allow changing of at least one of chemical and mechanical properties of the scaffold.

3. The chemical absorber as claimed in claim 1, wherein the surface modification comprises a coating.

4. The chemical absorber as claimed in claimed 1, wherein the surface modification comprises functional groups added to the at least one surface of the lattices.

5. The chemical absorber as claimed in claim 4, wherein the functional group comprises at least one selected from the group consisting of: strong cation exchange groups; weak cation exchange groups; strong anion exchange groups; weak anion exchange groups; DNA strands; biological ligands; proteins; antibodies; enzymes; nano-particles; micro-particles; magnetic particles; and any combination thereof.

6. The chemical absorber as claimed in claim 1, wherein the surface modification comprises a surface modification having multiple layers.

7. The chemical absorber as claimed in claim 1, wherein the dimension, shape, and mechanical properties of the absorber are determined by a size of vessels, location, and blood flow in a desired location for the absorber.

8. The chemical absorber as claimed in claim 1, wherein the absorber includes a sheath and a guide wire.

9. The chemical absorber as claimed in claim 8, wherein surfaces of the sheath and guide wire also have surface modifications.

10. The chemical absorber as claimed in claim 1, wherein the lattice and the scaffold are comprised of poly(ethylene glycol)-based polymers.

11. The chemical absorber of claim 1, wherein the lattice has a geometry comprised of at least one of: cube-like, hexagonal, a quasi-periodic structure, an aperiodic structure with different geometries at the front and back, and a continuously changing geometry wherein the changing geometry is one of either radial or axial, with wider struts at some locations and narrower struts at other locations.

12. A method of manufacturing a chemical absorber, comprising:
forming a three-dimensional porous scaffold of lattices having a center hole; and
modifying at least one surface of at least one of the scaffold and lattices to introduce functional groups, wherein the functional groups are selected based upon an ability to bond with a particular compound.

13. The method of manufacturing as claimed in claim 12, wherein forming the three-dimensional porous scaffold of lattices comprises forming the three-dimensional porous scaffold of lattices using elastomeric materials.

14. The method of manufacturing as claimed in claim 12, wherein forming the three-dimensional porous scaffold of lattices comprises forming the three-dimensional porous scaffold of lattices using poly(ethylene glycol)-based polymers.

15. The method of manufacturing as claimed in claim 12, wherein forming the three-dimensional porous scaffold of lattices comprises one of printing or injection molding the porous scaffold.

16. The method of manufacturing as claimed in claim 12, wherein modifying at least one surface comprises modifying the at least one surface through a chemical reaction.

17. The method of manufacturing as claimed in claim 16, wherein modifying the at least one surface through a chemical reaction comprises modifying the at least one surface through one of polymerization, catalytic reactions, surface coatings, etching, dopamine coating, and cross-linking.

18. The method of manufacturing as claimed in claim 12, wherein the functional groups are selected from the group consisting of: strong cation exchange groups; weak cation exchange groups; strong anion exchange groups; weak anion exchange groups; DNA strands; biological ligands; proteins; antibodies; enzymes; nano-particles; micro-particles; magnetic particles; and any combination thereof.

19. The method of manufacturing as claimed in claim 12, wherein the modifying the at least one surface comprises coating the at least one surface with polymers comprises of at least one of a combination of a first polymer to contain the functional groups and a second polymer to adhere the first polymer to the scaffold, a random copolymer comprising functional and adhering monomers, or graft copolymer comprising function and adhering monomers.

20. The method as claimed in claim 12, further comprising attaching the absorber to a guide wire and enclosing the absorber in a sheath.

21. The method as claimed in claim 20, further comprising modifying the surfaces of at least one of the guide wire and the sheath.

22. The method as claimed in claim 12, wherein forming the three-dimensional porous scaffold of lattices comprises forming the three-dimensional porous scaffold with dimensions, shape and mechanical properties based upon the size of vessels, location and blood flow rate in a desired location.

23. A method of introducing a chemical absorber into a body, comprising:
inserting a guide wire through a central hole in the chemical absorber;
attaching the chemical absorber to the guide wire;
inserting the chemical absorber into an introducer sheath;
inserting the introducing sheath into a blood vessel of the body; and
placing the chemical absorber into a vein from which blood from a tumor is drained.
